(19) United States
(12) Patent Application Publication
(10) Pub. No.: US 2004/0243107 A1
(43) Pub. Date: Dec. 2, 2004

(54) Methods and devices for treating atrial fibrilation

(76) Inventors: John A Macoviak, La Jolla, CA (US); David A Rahdert, San Francisco, CA (US)

Correspondence Address: RYAN KROMHOLZ & MANION, S.C., POST OFFICE BOX 26618, MILWAUKEE, WI, 53226, US

(21) Appl. No.: 10/491,461

(22) PCT Filed: Oct. 1, 2002
(86) PCT No.: PCT/US02/31374

(30) Foreign Application Priority Data
Oct. 1, 2001 (US) 60326590

(51) Int. Cl. A61B 17/00; A61B 19/00

(52) U.S. Cl. 606/1; 128/898

(57) ABSTRACT

The devices of the present invention form a platform, or scaffold for the precise delivery of various forms of energy for treatment of atrial fibrilation. Additionally, the devices of the present invention form a scaffold for the precise delivery of fluids to surrounding tissues. The use of additional energy sources can improve the delivery of various fluids into the surrounding tissue.

### REFERENCE TO PENDING PRIOR PATENT APPLICATION

This patent application claims benefit of pending prior U.S. Provisional Patent Application Ser. No. 60/326,590 filed Oct. 1, 2001 by John A. Macoviak, which patent is hereby incorporated by reference.

### FIELD OF THE INVENTION

This invention relates to methods and devices to improve the function of the heart. More particularly, the invention relates to methods and devices to treat atrial fibrillation.

### BACKGROUND OF THE INVENTION

To function properly as a pump, the heart must contract in a rhythmic pattern. Heart rhythm is normally established at a single point called the sinoatrial node, or SA node, located in the right atrium of the heart, near the opening of the superior vena cava. The SA node generates electrical impulses which spread throughout the heart and result in a rhythmic contraction of the heart, termed a sinus rhythm. Thus, the SA node functions as a pacemaker for the heart.

Other regions of the heart can potentially produce electrical impulses. A pacemaker other than the SA node is referred to as an ectopic pacemaker. Electrical signals from an ectopic pacemaker can disrupt a rhythmically contracting heart, resulting in an arrhythmia, characterized by a chaotic, disorganized heart rhythm. Fibrillation of the atria results in loss of atrial contraction and rapid impulses being sent to the ventricles causing high and irregular heart rates.

Atrial fibrillation (AF) is clinically related to several conditions, including anxiety, increased risk of stroke, reduced exercise tolerance, cardiomyopathy, congestive heart failure and decreased survival. Patients who experience AF are, generally, acutely aware of the symptoms.

Current curative AF therapies are based upon a procedure that has become known as the Cox Maze procedure. The Cox Maze procedure is an open-heart, surgical procedure that requires the patient to be placed on cardiopulmonary bypass equipment. The procedure requires six hours and the patient to be under general anesthesia. In this procedure, access to the heart is gained by way of a median sternotomy, which is a surgical split of the breast bone. The left atrium is surgically incised along predetermined lines known to be effective in blocking the transmission of electrical signals from an ectopic pacemaker that triggers AF. The incision lines create blocks that prevent conduction of unwanted electrical signals throughout the heart and permit a normal pattern of depolarization of the atria and ventricles beginning in the SA node and traveling to the AV or atrioventricular node.

Less invasive methods and devices for treating AF are needed that improve heart function and improve patient safety.

### SUMMARY OF THE INVENTION

The devices of the present invention form a platform, or scaffold for the precise delivery of various forms of energy for treatment of atrial fibrilation. Additionally, the devices of the present invention form a scaffold for the precise delivery of fluids to surrounding tissues. The use of additional energy sources can improve the delivery of various fluids into the surrounding tissue.

### BRIEF DESCRIPTION OF THE DRAWINGS

FIG. 1 shows an embodiment of the invention in relation to its position within the heart, and within a patient's body.

FIG. 2 shows an enlarged view of the device of FIG. 1, with the loops surrounding the outlet of the pulmonary veins 210.

FIG. 3 shows the reverse side of the device of FIGS. 1 and 2. The reverse side's loop section 320 is shown having a multitude of holes, or micro-ports 330, that lie adjacent to the atrial walls.

FIG. 4 shows an embodiment of the invention 400, in fluid communication with a catheter 410.

FIG. 5 show an embodiment of the device shown in FIG. 4.

FIG. 6 is a frontal view of the device of FIGS. 4 and 5, with an additional positioning element.

FIG. 7 is a longitudinal cross section of one embodiment of a tubule 720, having several micro-ports 730.

FIG. 8 shows a radial cross section of the tubule shown in FIG. 7.

FIGS. 9 and 10 show alternative tubule 910 designs, wherein the micro-ports are filled with porous plugs 920.

FIG. 111 shows a catheter being introduced from the inferior vena cava 1110, into the right atrium 1140, through a septum 1120 between the right and left atrium, and into the left atrium 1150.

FIG. 12 illustrates an embodiment of the invention 1200 that may be used to deliver energy to designated tissue.

FIG. 13 shows an embodiment of the invention, and the use of an energy source 1310 to deliver energy to devices of the present invention.

FIG. 14 shows an embodiment of the invention 1400 having a positioning structure 1410 to standardize scaffold orientation within a treated heart chamber.

FIG. 15 shows a scaffold in the form of a wire coil that, when deployed, closely conforms to the interior of a patient's heart chamber, such as the patient's left atrium in the example shown.

FIG. 16 shows another embodiment for the scaffold 1600 of present invention. The scaffold is in the form of a wire cage that, when deployed, closely conforms to the interior of a patient's heart chamber, such as the patient's left atrium.

FIG. 17 shows another embodiment for the scaffold 1700 of present invention.

FIG. 18 illustrates an alternative embodiment 1800 of the invention, positioned within the right atrium.

FIGS. 19 through 22 show various embodiments of the invention having dual chamber structures.

FIGS. 23-25 show schematic views of a patient with a catheter 2340 being advanced from the inferior vena cava 2330, into the right atrium, and across the septum into the left atrium. A second catheter 2320 is being advanced through the esophagus 2320.

### DETAILED DESCRIPTION

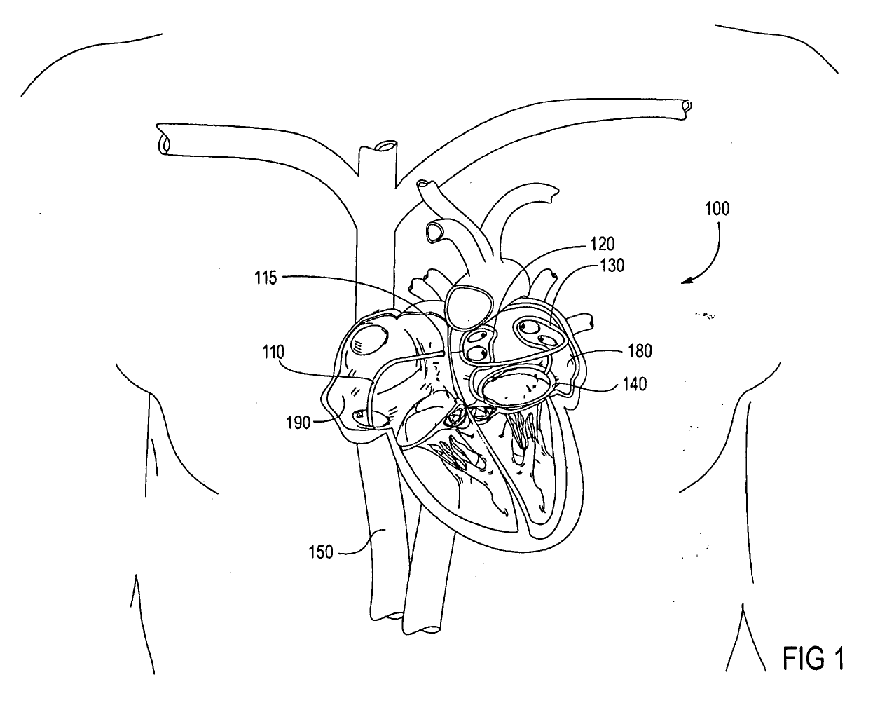

FIG. 1 shows an embodiment of the invention in relation to its position within the heart, and within a patient's body. The device 100 is comprised of a platform, or scaffold that is shown being introduced from the inferior vena cava 150, into the right atrium 190, across the septum 115 between the right and left atrium, and into the left atrium 180. The device 100 scaffold is shown having a right ablation loop 120, a left ablation loop 130, and an annular base 140. The right and left ablation loops are shown to come within close proximity of the atrial walls that surround the pulmonary veins. The pulmonary veins are common sources of ectopic pacemakers.

The device 100 is advanced through a catheter 110 and into position. Alternatively, the device 100 may be pre-loaded within a delivery catheter.

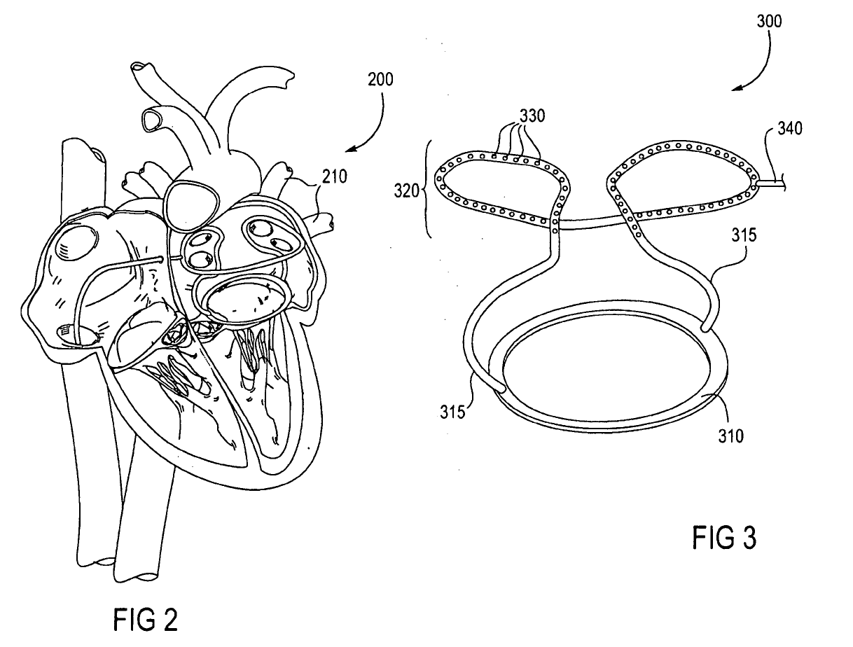

FIG. 2 shows an enlarged view of the device of FIG. 1, with the loops surrounding the outlet of the pulmonary veins 210. The device may be used as a temporary platform, or scaffold, from which therapeutic fluids or energy can be deployed. Alternatively, the device may be left in place as a permanent implant.

Although the device 200 may have a gap of incomplete contact between the device and target tissue, the device is still effective, as described below, especially when used conjunction with tissue disrupting energies (electroporation or sonoporation), energies that promote fluid flow (electrophoresis or sonophoresis), and energies that promote scaffold vibrations. Many types of energies can be delivered to the scaffold either directly, or indirectly. Indirect application (using non-contact means) of energies can be applied trans-esophageally, trans-bronchially, trans-tracheally, trans-thoracically, across the sternum, etc.

FIG. 3 shows the reverse side of the device of FIGS. 1 and 2. The reverse side's loop section 320 is shown having a multitude of holes, or micro-ports 330, that lie adjacent to the atrial walls. The micro-ports can be laser cut along the mural facing surface of the device. The micro-ports direct fluids within the device to be released into adjacent tissues. Fluids within the device may include alcohol, potassium iodide, therapeutic drugs, etc.

Alternatively, the devices of the present invention may not have any micro-ports, and instead be used as a heat exchanger. For example, a heat removing fluid could be circulated within the device, thus giving rise to a temporary conduction block in the adjacent tissue. As such, the device 300 can be used a diagnostic tool, for determining the origin of ectopic pacemakers, for example. Also, with longer exposures to adjacent tissues, the heat removal aspect of the device could result in permanent conduction block, tissue shrinkage (to tighten the skin, for promoting valve function, or close off an atrial appendage), etc.

When used in the left atrium, the device's annular base 310 is positioned to surround the mitral annulus. The loop section 320 is supported by upright members 315. The loop section 320 is in fluid communication with the catheter via the inlet port 340.

As shown, this device may be used to prevent AF, but in a manner that differs from the Cox Maze procedure. In the Cox Maze procedure, a specific pattern is cut into the heart to create a proper pathway for the signal generated from the SA node to travel throughout the heart. The device shown differs in that it does not create a signal pathway, but rather isolates unwanted signals from propagating. The procedure is intended for use by an interventional electro-cardiologist, or other skilled professional.

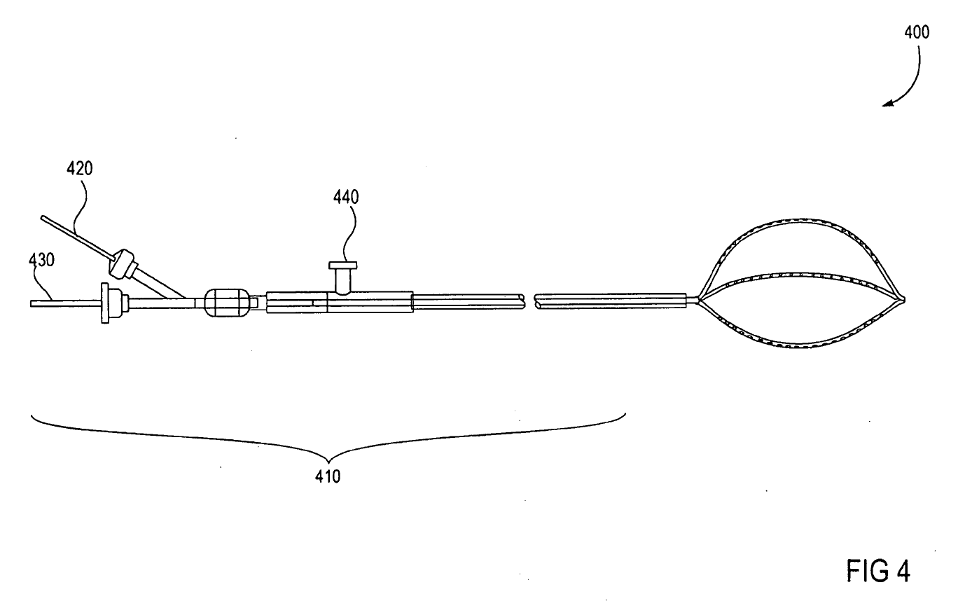

FIG. 4 shows an embodiment of the invention 400, in fluid communication with a catheter 410. The catheter 410 may be introduced into the femoral vein, and advanced through the vena cava into the right atrium. The catheter may be 12 to 14 French in diameter and approximately 150 centimeters long, depending on the dimensions of the patient's anatomy. An exemplary catheter 410 is shown to have a guide wire port 420, a thru lumen port 430, and an ablation agent vent 440. Not shown is an ablation agent inlet port. Preferred ablation agents are alcohol, or potassium iodide.

The catheter may be introduced into the patient under fluoroscopic guidance and advanced through the venous return to the right atrium of the heart. Using standard cardiology procedures, a trans-septal puncture will be performed and the catheter 410 may be advanced through the trans-septal puncture into the left atrium. Guide wires may be advanced into the atrial appendage, the mitral valve annulus and one of the pulmonary veins. The device is preferably designed from a biocompatible, super-elastic material that will expand aggressively under the effects of body heat, or with the aid of an inflatable balloon. Under continued fluoroscopic guidance with the adjunctive capability for verification by intravascular ultrasound, the cardiologist will ensure that the device has expanded completely, and is positioned correctly and in close contact with surrounding heart wall. The device is then used as a platform for the delivery of energy or a fluid that can create a conduction block, or be used diagnostically. Conduction block lines preferably fully transect the myocardium of the atrium (about 3 to 5 millimeters in thickness). Once the conduction block has been completed, the device may be removed from the patient.

The benefits of using alcohol, or other tissue fixative agents, is the drastic reduction of energy required to create conduction block, resulting in a safer and more effective ablation because the tissue is in fact toughened by the fixative properties of alcohol-like agents that cause a coagulation cellular necrosis instead of a weakened tissue wall liquefaction necrosis that is caused with other types of energy to create conduction block.

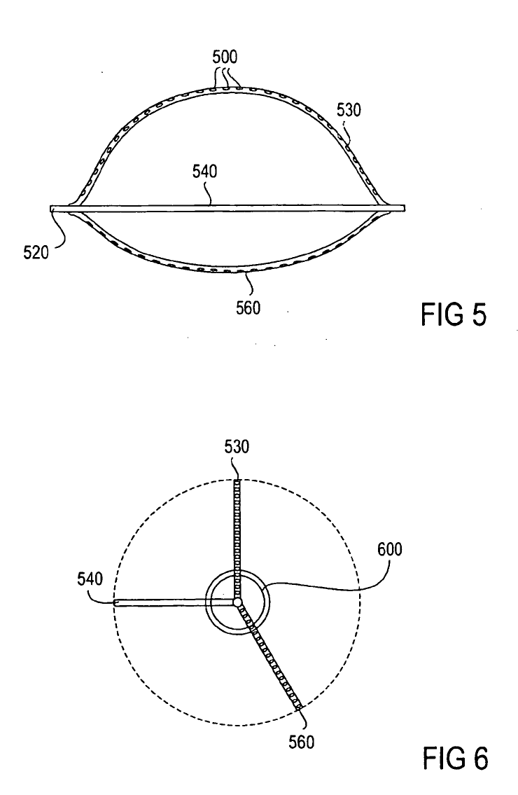

FIG. 5 show an embodiment of the device shown in FIG. 4. The device is shown with an opposition member 540, a superior tubule 530 (superior relative to the pulmonary veins), and an inferior tubule 560 (inferior relative to the pulmonary veins). In addition, the device can be designed with additional tubules to create additional lines of ablation, or additional opposition members. Assuming a trans-septal introduction of this embodiment from the right atrium into the left atrium, the proximal end 520 of the device is positioned adjacent the trans-septal entry point. The opposition member 540 is positioned along the anterior wall, opposite the pulmonary veins. The opposition member functions to transmit mural pressure from the atrium through the device to the tubules. The superior tubule, 530, is positioned adjacent the apex of the left atrium. The inferior tubule, 560, is positioned adjacent the base of the posterior wall. The tubules, 530 and 560, have a multitude of micro-ports 500. The micro-ports allow a fluid to be released from inside the tubules and into the atrial walls. Several fluids can be used, any of which function to disrupt the flow of unwanted electrical signals. Thus, the fluids released from the micro-ports located along the tubules create an electrical signal block. The shape of signal block created by this embodiment is that of an oval, or a football. The lines follow a path similar to two adjacent longitudinal lines on a world globe (turned sideways) beginning at the North Pole, and ending at the South Pole.

FIG. 6 is a frontal view of the device of FIGS. 4 and 5. An additional aspect of the device includes an orienting structure, so that the device takes advantage of anatomical features to achieve proper orientation within a heart chamber. For example, FIG. 6 shows a circular structure 600 projecting from the distal end of the device. This circular projection may be positioned within an atrial appendage to aid with orientation of the device. This may be designed in the shaped of a pigtail, or corkscrew projecting from the distal end of the device.

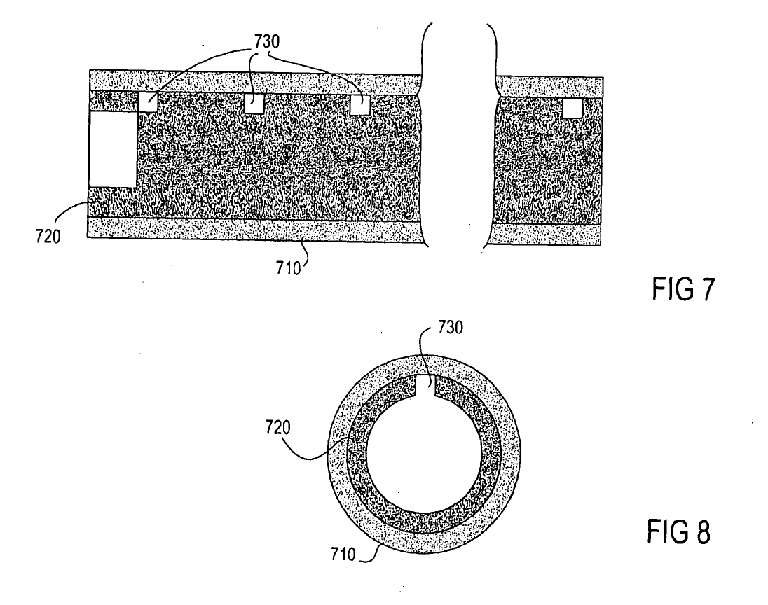

FIG. 7 is a longitudinal cross section of one embodiment of a tubule 720, having several micro-ports 730. The tubule 720 is encased within a sleeve 710. A preferred sleeve 710 is a polymeric sleeve made from sintered gel. The sleeve 710, functions as a diffusion barrier so that when fluid is released from the tubule 720, it is slowed down and allowed to diffuse into the adjacent atrial wall, rather than be released like a jet into the surrounding atrial wall. The sleeve 710 also promotes an equal distribution of fluid throughout the tubule 720.

FIG. 8 shows a radial cross section of the tubule shown in FIG. 7. Nitinol is a material that may be used for the tubule 720.

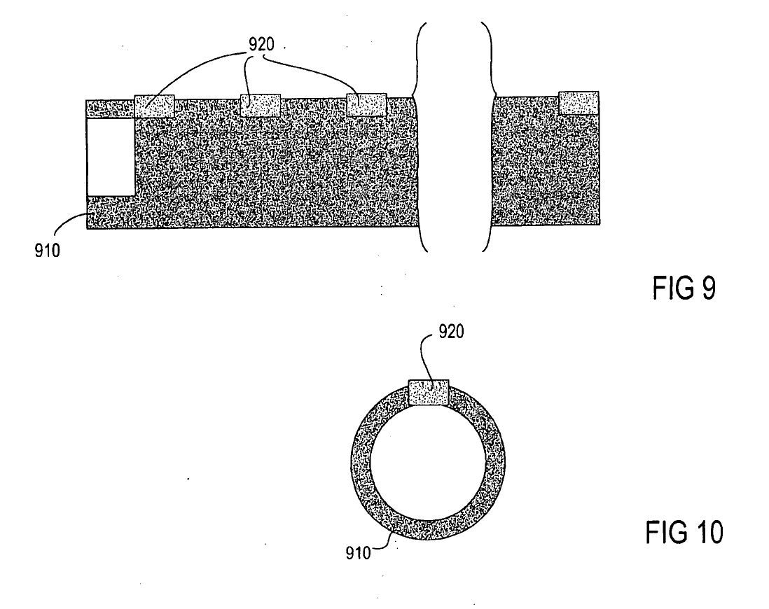

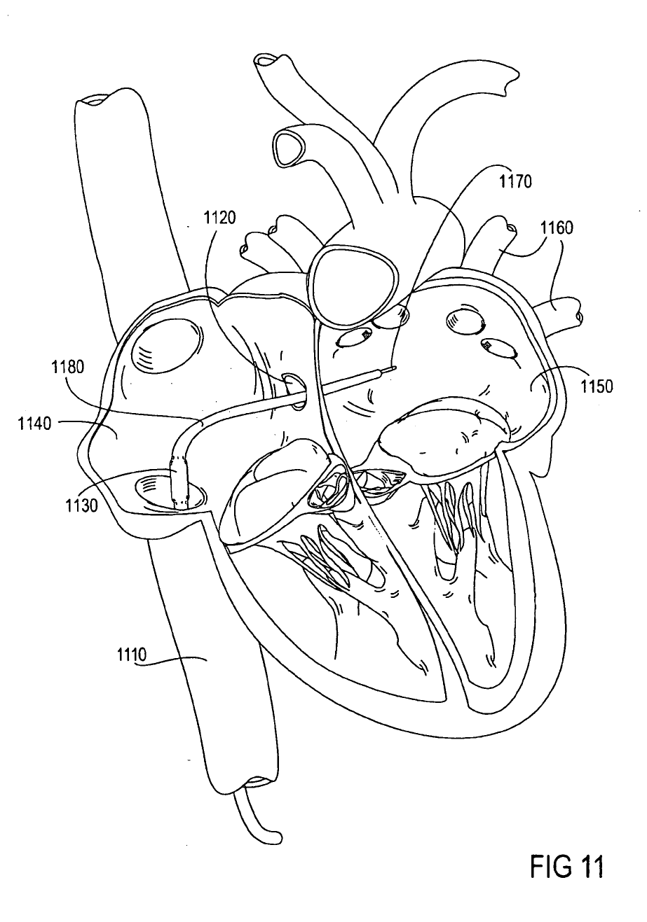

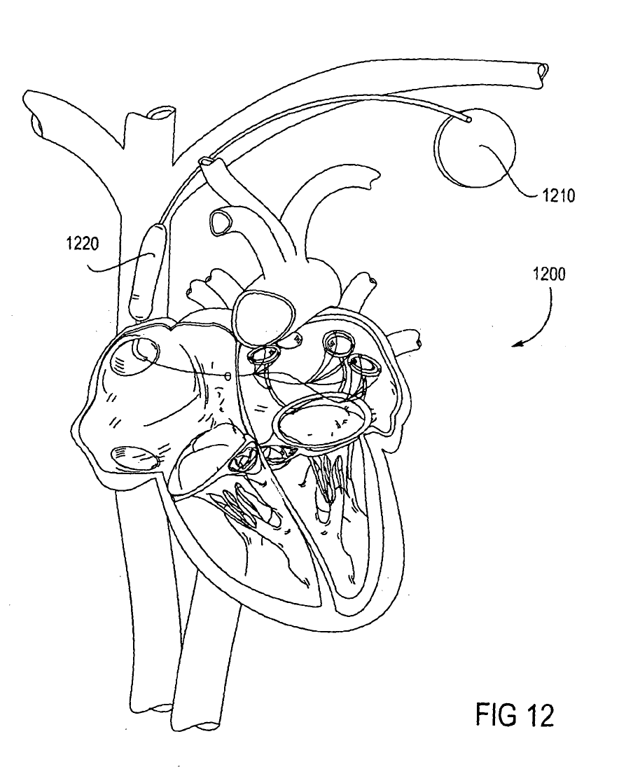

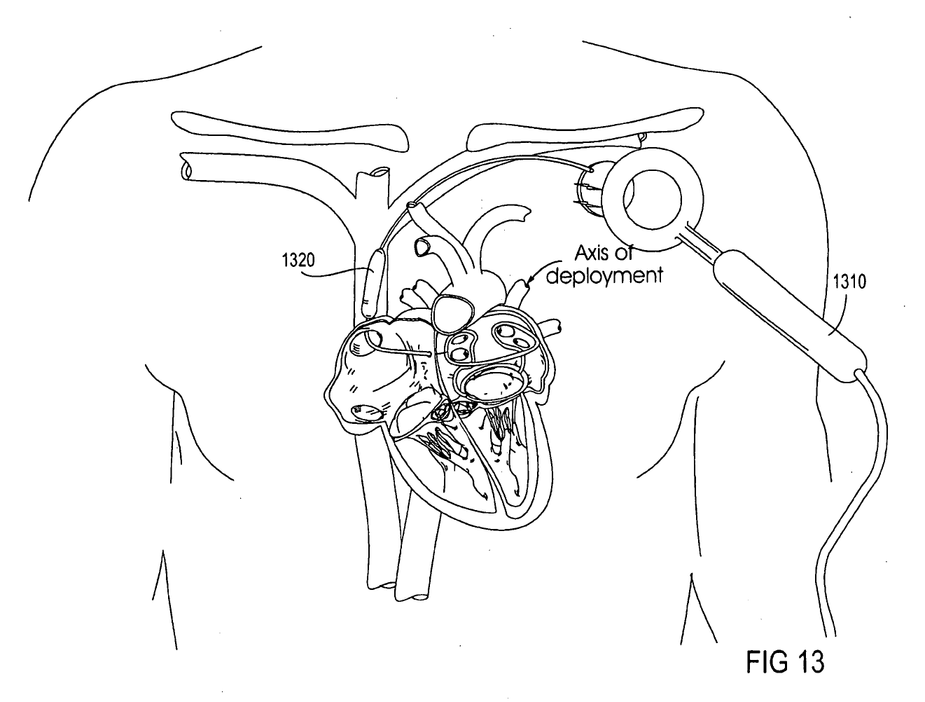

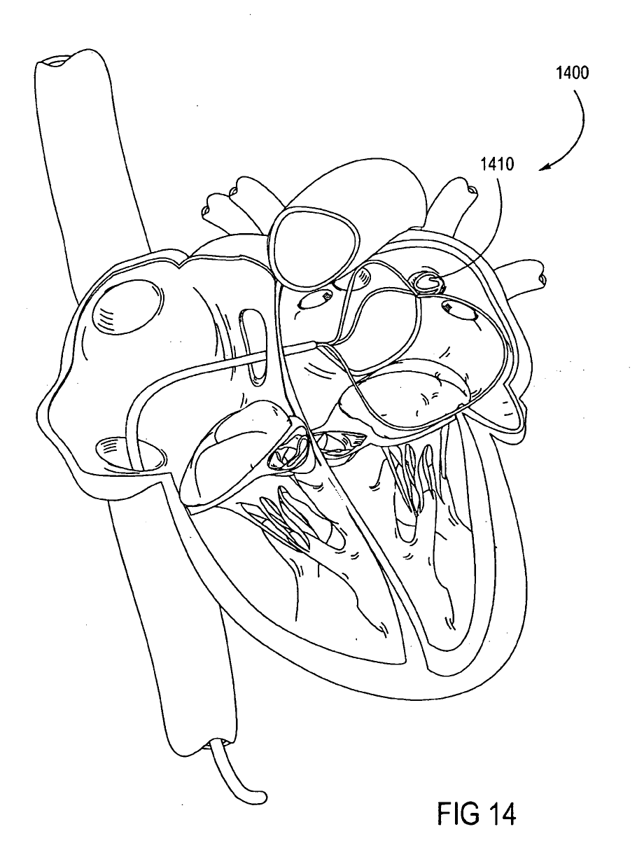

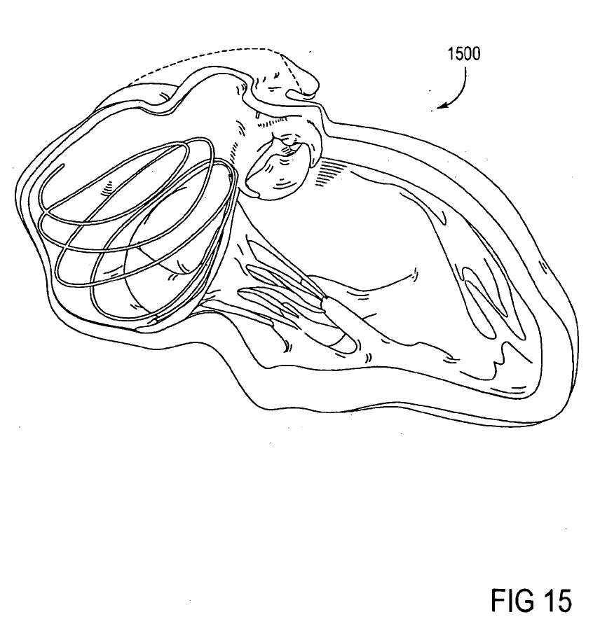

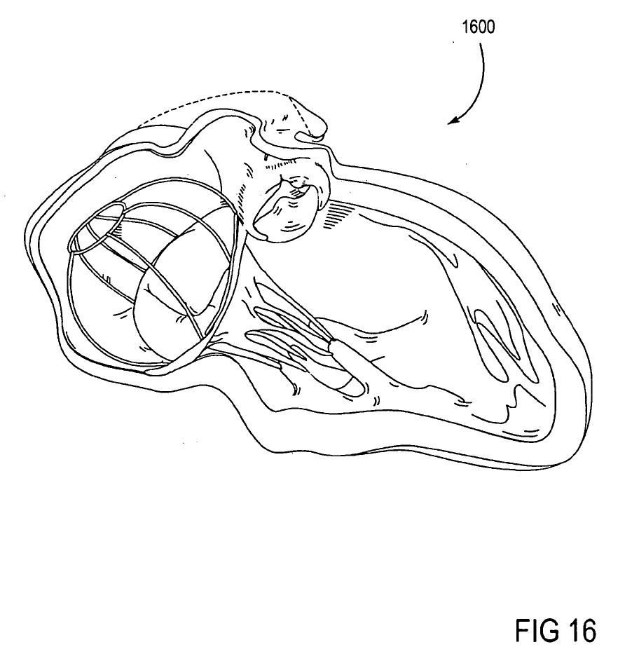

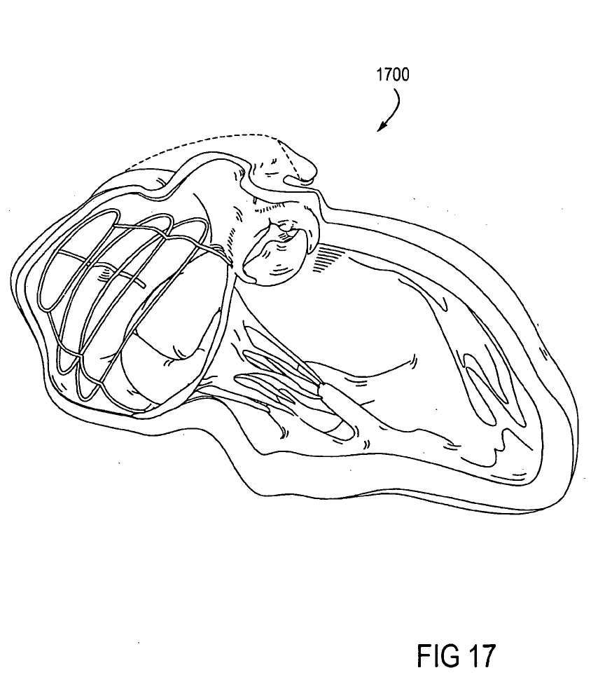

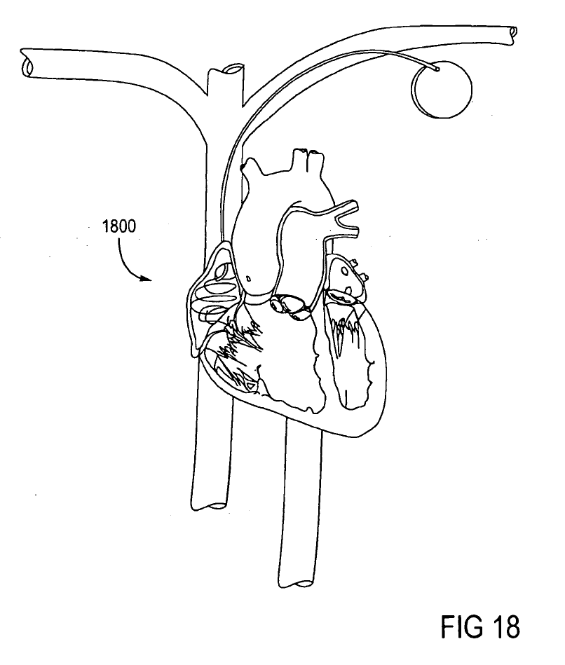

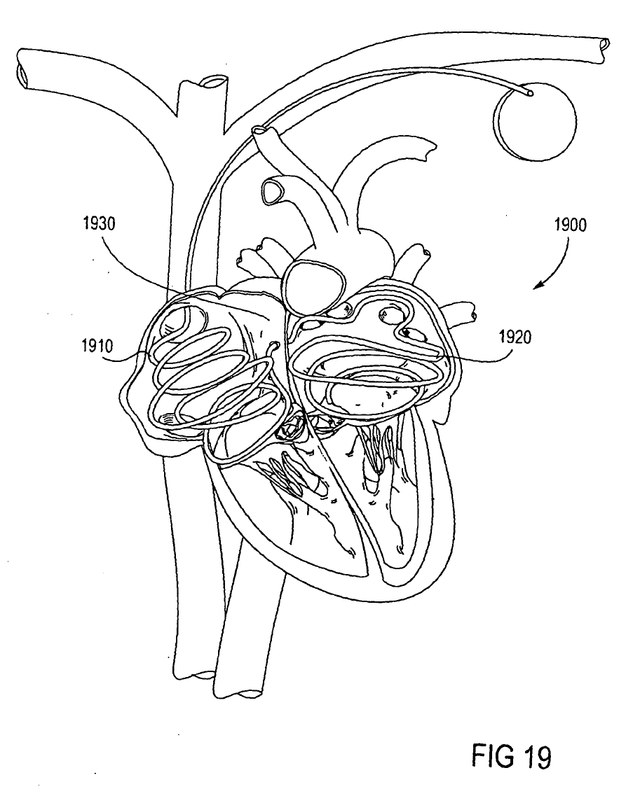

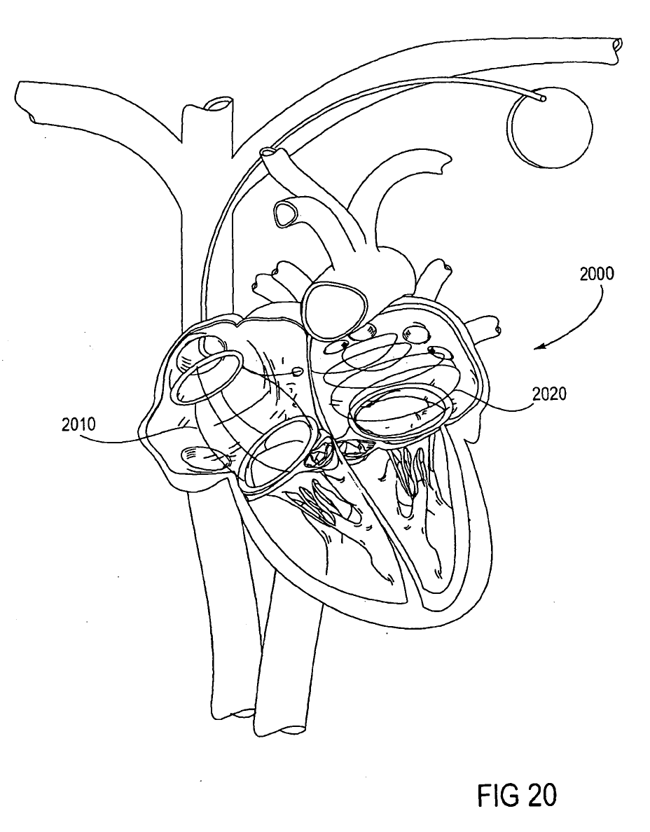

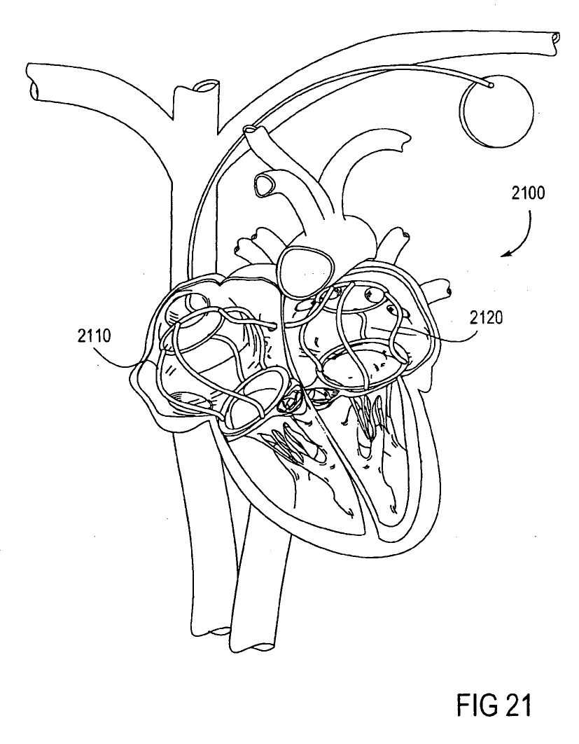

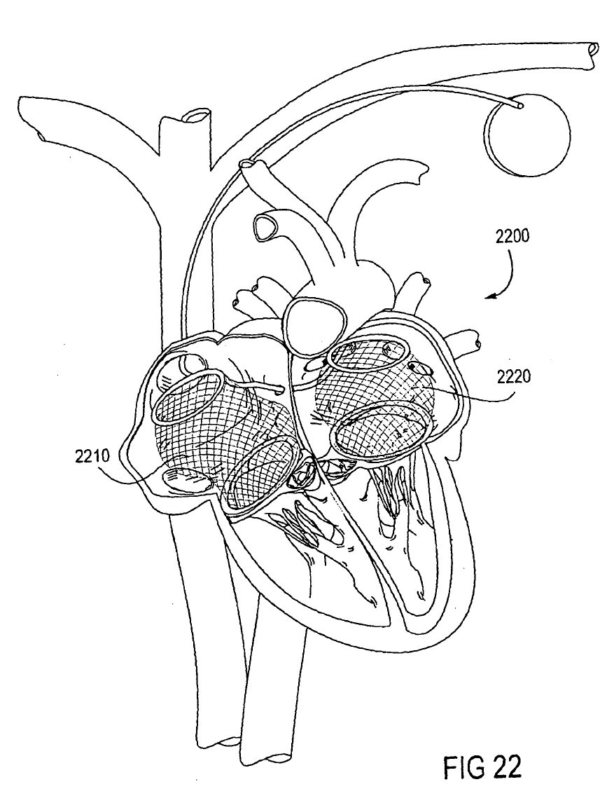

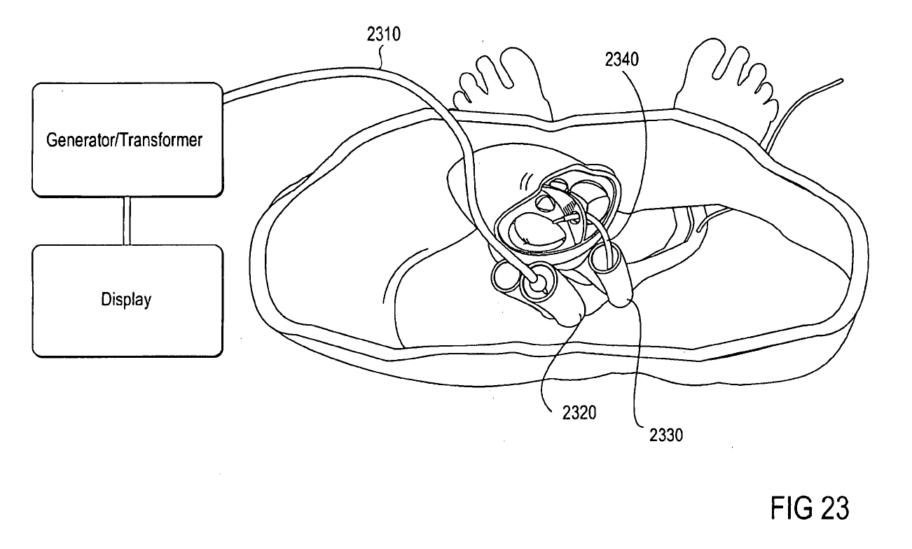

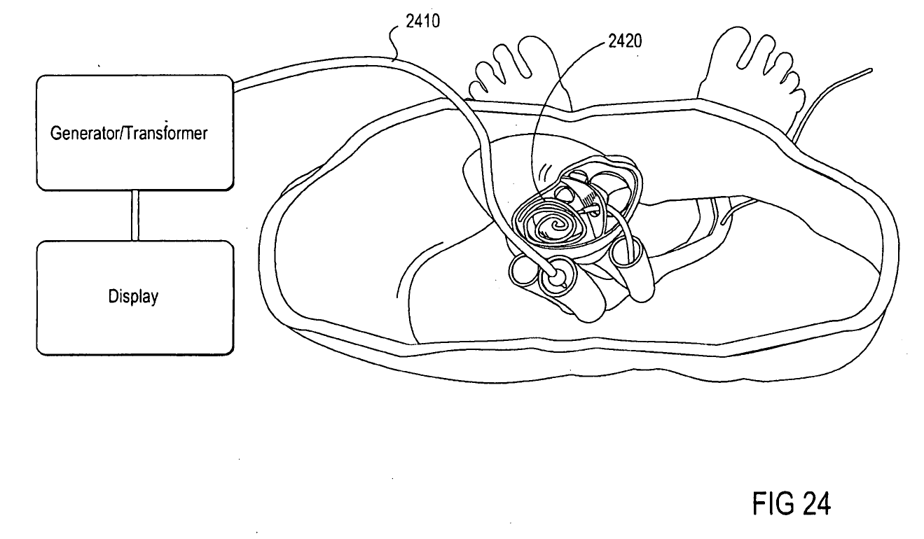

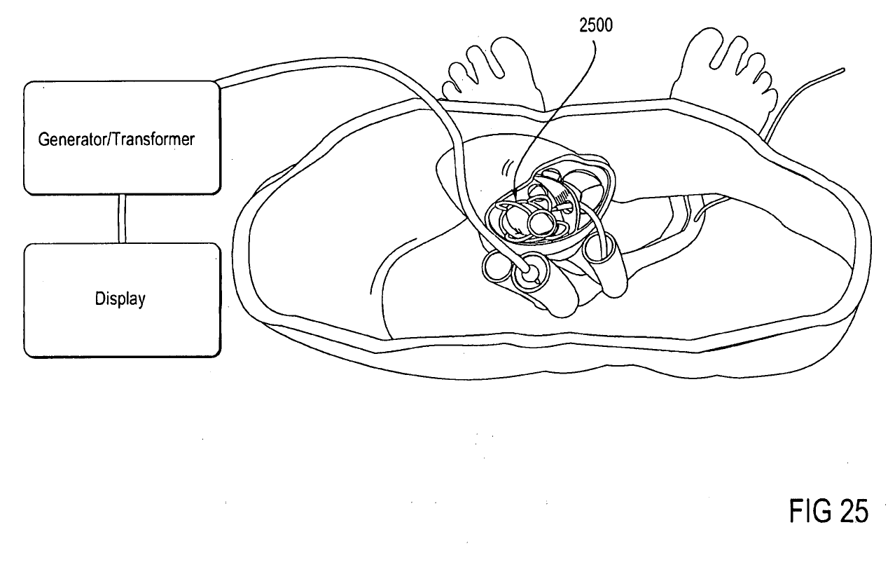

FIGS. 9 and 10 show alternative tubule 910 designs, wherein the micro-ports are filled with porous plugs 920. A preferred porous plug 920 is comprised of sintered gel beads formed into a porous plug.

FIG. 11 shows a catheter being introduced from the inferior vena cava 1110, into the right atrium 1140, through a septum 1120 between the right and left atrium, and into the left atrium 1150. This figure illustrates a pump 1130 positioned within a catheter 1180. Also, there is a guide wire 1170 shown protruding from the distal end of the catheter 1180. The pump 1130 may be a piezoelectric pump used to drive fluid out through the micro-ports of the tubules. In another embodiment, there may be no in-line pump. Instead, an outside pump may be used.

FIG. 12 illustrates an embodiment of the invention 1200 that may be used to deliver energy to designated tissue. The device is shown connected to an energy component 1210 that may be a generator, defibrillator, pacemaker, or radio frequency device, that has been positioned underneath the skin (subclavian pocket) and that makes its way into the superior vena cava via the subclavian vein. The device structure 1220 shown within the superior vena cava may function as a transformer, capacitor, or electrode.

FIG. 13 shows an embodiment of the invention, and the use of an energy source 1310 to deliver energy to devices of the present invention. The in-line member 1320 could be a transformer, capacitor, or electrode, depending on the need.

FIG. 14 shows an embodiment of the invention 1400 having a positioning structure 1410 to standardize scaffold orientation within a treated heart chamber. In this embodiment, the positioning structure 1410 is shown being introduced to a pulmonary vein.

FIGS. 15 through 18 illustrate various embodiments of the invention.

FIG. 15 shows a scaffold in the form of a wire coil that, when deployed, closely conforms to the interior of a patient's heart chamber, such as the patient's left atrium in the example shown. The deployed scaffold has an approximately cylindrical configuration. The wire coil of the scaffold may be constructed of a malleable or elastic biocompatible metal, such as stainless steel or a super-elastic or shape memory nickel/titanium alloy, for example. Preferably, the scaffold is sufficiently flexible such that it does not interfere with the normal contraction of the heart. In addition, the wire coil may have a coating for improved biocompatibility, thermal and/or electrical insulation, etc.

FIG. 16 shows another embodiment for the scaffold 1600 of present invention. The scaffold is in the form of a wire cage that, when deployed, closely conforms to the interior of a patient's heart chamber, such as the patient's left atrium. The deployed scaffold may have a dome-shaped or tapered cylindrical configuration, with an upper loop and a lower loop joined by longitudinal struts.

FIG. 17 shows another embodiment for the scaffold 1700 of present invention. The scaffold is in the form of a hoop-and-strut wire cage that, when deployed, closely conforms to the interior of a patient's heart chamber, such as the patient's left atrium. The deployed scaffold may have a dome-shaped or tapered cylindrical configuration, with an upper hoop, a middle hoop and a lower hoop joined by longitudinal struts.

FIGS. 19 through 22 show various embodiments of the invention having dual chamber structures.

FIGS. 23-25 show schematic views of a patient with a catheter 2340 being advanced from the inferior vena cava 2330, into the right atrium, and across the septum into the left atrium. A second catheter 2320 is being advanced through the esophagus 2320, and its close proximity to the left atrium makes it a suitable pathway for delivering a non-contact energy source, such as ultrasound (preferably low frequency ultrasound, below 1 MHz), radio frequency, or an inductive coupling mechanism. Alternative non-contact energy source include microwaves. These energy sources can be applied to various devices to encourage the flow of ions in a preferred direction, encourage fluid absorption, or cause ablation to occur. Also, ultrasound and other energy sources may be delivered to the devices of the present invention across the skin, transcutaneously.

While the present invention has been described herein with respect to the exemplary embodiments and the best mode for practicing the invention it will become apparent to one of ordinary skill in the art that many modifications, improvements and sub combinations of the various embodiments, adaptations and variations can be made to the invention without departing from the spirit and scope thereof.

We claim:

1. A platform scaffold for treating atrial fibrilation, the platform scaffold comprising:
an annular base,
a tubular loop section,
upright members,
and an inlet port,
wherein the annular base supports the loop section via the upright members, and the inlet port enables communication with other devices.

2. The platform scaffold of claim 1, further comprising micro-ports positioned along the heart wall contacting surface of the tubular loop section.

3. The platform scaffold of claim 2, wherein the micro-ports are filled with porous plugs.

4. The platform scaffold of claim 1, wherein the platform scaffold is manufactured from a super-elastic material.

5. The platform scaffold of claim 1, wherein the tubular loop section is encased within a polymeric sleeve.

6. A platform scaffold for treating atrial fibrilation, the platform scaffold comprising:
a first structural rail,
a second therapeutic rail,
and a third therapeutic rail,
wherein the rails originate at a first point, and terminate at a second point, and expand radially away from the first point, and contract radially toward the second point, and when placed within a heart chamber, the first structural member transmits force from the surrounding heart wall to the second and third therapeutic rails to ensure all rails contact the adjacent heart wall.

7. The second and third therapeutic rails of claim 6, further comprising micro-ports positioned along the heart wall contacting surface of the rails.

8. The second and third therapeutic rails of claim 7, wherein the micro-ports are filled with porous plugs.

9. The platform scaffold claim 6, wherein the platform scaffold is manufactured from a super-elastic material.

10. The platform scaffold of claim 6, wherein the rails are encased within a polymeric sleeve.

11. The platform scaffold of claim 6, further comprising a positioning member to standardize scaffold orientation within a treated heart chamber.

12. A platform scaffold for treating atrial fibrilation, comprising:
a wire, the wire having an approximately cylindrical configuration when deployed closely conforming to the interior of a patient's heart chamber.

13. The platform scaffold of claim 12, wherein the platform scaffold is manufactured from a super-elastic material.

14. A platform scaffold for treating atrial fibrilation, comprising:
a wire form birdcage, the wire form birdcage having a dome-shaped or tapered cylindrical configuration, with an upper loop and a lower loop joined by longitudinal struts, the wire form birdcage closely conforming to the interior of a patient's heart chamber.

15. The platform scaffold of claim 14, wherein the platform scaffold is manufactured from a super-elastic material.

16. A platform scaffold for treating atrial fibrilation, comprising:
a wire form hoop-and-strut wire cage, the wire form hoop-and-strut wire cage having a dome-shaped or tapered cylindrical configuration, with an upper hoop, a middle hoop and a lower hoop joined by longitudinal struts, the wire form hoop-and-strut wire cage closely conforming to the interior of a patient's heart chamber.

17. The platform scaffold of claim 16, wherein the platform scaffold is manufactured from a super-elastic material.

18. A method of diagnosing signal conduction within the heart comprising:
transvascularly introducing a platform scaffold into the heart;
positioning the platform scaffold;
releasing a heat absorbing fluid into the platform scaffold, the heat absorbing fluid them passing through the platform scaffold;
and observing the effect of localized cooling on the heart to temporarily interrupt signal conduction.

19. A method of creating a signal block within the heart comprising:
transvascularly introducing a platform scaffold into the heart having micro-ports;
positioning the platform scaffold;
and releasing a tissue fixative fluid into the scaffold, the tissue fixative fluid then passing through the platform scaffold.

20. The method of creating a signal block within the heart of claim 19, further comprising the step of applying tissue disrupting energies that promote fluid flow.

21. The method of creating a signal block within the heart of claim 20, wherein the tissue disrupting energy may be applied directly to the scaffold.

22. The method of creating a signal block within the heart of claim 20, wherein the tissue disrupting energy may be applied indirectly to the scaffold.

23. The method of creating a signal block within the heart of claim 19, further comprising the step of applying energies that promote scaffold vibrations.

24. The method of creating a signal block within the heart of claim 23, wherein the energies that promote scaffold vibrations may be applied directly to the scaffold.

25. The method of creating a signal block within the heart of claim 23, wherein the energies that promote scaffold vibrations may be applied indirectly to the scaffold.

26. A method of creating a signal block within the heart comprising:
transvascularly introducing a platform scaffold into the heart;
positioning the platform scaffold;
and transferring energy to the scaffold to create lines of ablation.

27. The method of creating a signal block within the heart of claim 26, wherein the energies that creates lines of ablation may be applied directly to the scaffold.

28. The method of creating a signal block within the heart of claim 26, wherein the energies that create lines of ablation may be applied indirectly to the scaffold.
